(19) United States
(12) Patent Application Publication
(10) Pub. No.: US 2020/0060820 A1
(43) Pub. Date: Feb. 27, 2020

(54) SYSTEMS AND METHODS FOR POSITIONING MEDICAL DEVICES RELATIVE TO HEART VALVE ANATOMY

(71) Applicant: Mitralix Ltd., Yokne'am Moshava (IL)

(72) Inventors: Yonatan Ben-Zvi, Kiryat Tiv'on (IL); Noam Weiss, Tel Aviv (IL); Ira Yaron, Har Adar (IL)

(21) Appl. No.: 16/567,235

(22) Filed: Sep. 11, 2019

Related U.S. Application Data
(60) Provisional application No. 62/734,022, filed on Sep. 20, 2018.

(51) Int. Cl. A61F 2/24; A61B 17/00

(52) U.S. Cl. CPC A61F 2/2427; A61F 2002/9517; A61B 17/00234

(57) ABSTRACT

Example systems and methods for positioning a medical device relative to a tricuspid valve and a mitral valve are disclosed. Example methods comprise selecting the desired positioning and performing predefined steps involving turning the control knob of a steerable introducer in a predetermined direction and/or rotating the flexible tubular catheter of the steerable introducer around its longitudinal axis in a predetermined direction. Example methods also comprise selecting the desired positioning and performing predefined steps involving turning the control knob of a steerable introducer in a predetermined direction by a predetermined amount and/or rotating the flexible tubular catheter of the steerable introducer around its longitudinal axis in a predetermined direction by a predetermined amount. Example methods include following a road map of steps to a desired position relative to a tricuspid valve or a mitral valve.

### CROSS-REFERENCE TO RELATED APPLICATION

This application claims the priority benefit of U.S. provisional application Ser. No. 62/734,022, filed on Sep. 20, 2018.

### FIELD OF THE INVENTION

This disclosure relates generally to systems and methods for positioning medical devices relative to heart valve anatomy.

### BACKGROUND OF THE INVENTION

Various medical procedures are performed inside of a patient's heart using a steerable introducer to position a medical device to a desired location in the heart for performing the procedure. For example, a number of techniques involve threading a steerable introducer through the patient's vasculature and into the heart and deploying the medical device through the steerable introducer. In some procedures, for example, a steerable introducer may be inserted percutaneously into a patient's vein or artery, such as patient's subclavian vein, internal jugular vein, or femoral artery or vein, and the steerable introducer may be advanced from there through the patient's vasculature into the patient's heart.

Procedures performed inside the heart include, for example, heart valve repair and heart valve replacement. In one example of heart valve repair, a heart valve repair device is delivered by a delivery system that is tracked to the heart inside of the steerable introducer. Many other procedures are performed inside the heart, such as, for example, ablation of tissue for treatment of atrial fibrillation. An ablation instrument or other instrument may be tracked to the heart inside of the steerable introducer.

In many of these procedures, it is desirable to position the medical device in a desired location relative to a heart valve. For example, it may be desired to position a heart valve repair device in a desired location relative to the leaflets of the tricuspid valve or the mitral valve.

In certain prior procedures, the positioning of a medical device (e.g., a delivery system for an implant or an ablation catheter) is performed by an operator (e.g., a physician) assessing the location of the steerable introducer by use of a visualization technique such as echocardiography. The operator assesses the positioning by visualization and maneuvers the steerable introducer, making adjustments in accordance with the visual feedback in order to move to the desired position. Such methods can be time-consuming and inefficient.

A need exists for improved systems and methods for positioning medical devices relative to heart valve anatomy that reduce procedure time and risk, reduce cost, improve ease of use, and/or improve overall clinical outcome.

### SUMMARY OF THE INVENTION

The disclosure provides various systems and methods for positioning medical devices relative to heart valve anatomy.

Example systems and methods for positioning a medical device relative to a tricuspid valve and a mitral valve are disclosed. Example methods comprise selecting the desired positioning and performing predefined steps involving turning the control knob of a steerable introducer in a predetermined direction and/or rotating the flexible tubular catheter of the steerable introducer around its longitudinal axis in a predetermined direction. Example methods also comprise selecting the desired positioning and performing predefined steps involving turning the control knob of a steerable introducer in a predetermined direction by a predetermined amount and/or rotating the flexible tubular catheter of the steerable introducer around its longitudinal axis in a predetermined direction by a predetermined amount.

An example method for positioning a medical device relative to heart valve anatomy of a patient comprises the steps of: inserting a steerable introducer into the patient, the steerable introducer comprising (a) a flexible tubular catheter comprising a longitudinal axis and distal tip and (b) a control knob for bending and straightening the distal tip; inserting a medical device through the steerable introducer; moving the distal tip of the flexible tubular catheter into approximately a center of the right atrium of the heart of the patient; adjusting the distal tip of the flexible tubular catheter to face the tricuspid valve of the patient between the right atrium and the right ventricle; and operating the steerable introducer to move the distal tip toward a desired valve area in a manner selected from the group of: (i) to move the distal tip toward an area of the tricuspid valve commissure between the anterior leaflet and the posterior leaflet of the tricuspid valve, rotating the flexible tubular catheter around its longitudinal axis in a counterclockwise direction when viewed distally, for example by about one-quarter turn, (ii) to move the distal tip toward an area of the tricuspid valve commissure between the posterior leaflet and the septal leaflet of the tricuspid valve, rotating the flexible tubular catheter around its longitudinal axis in a clockwise direction when viewed distally, for example by about one-eighth turn, and turning the control knob in a direction of bending the distal tip of the flexible tubular catheter, for example by about one-quarter turn of the control knob, and (iii) to move the distal tip toward an area of the tricuspid valve commissure between the septal leaflet and the anterior leaflet of the tricuspid valve, turning the control knob in a direction of straightening the distal tip of the flexible tubular catheter, for example by about one-quarter turn of the control knob.

The step of adjusting the distal tip of the flexible tubular catheter to face the tricuspid valve may comprise turning the control knob in a direction of bending the distal tip of the flexible tubular catheter, for example by about one-quarter turn to about one-half turn of the control knob.

The step of inserting the medical device through the steerable introducer may be performed before or after the step of inserting the steerable introducer into the patient.

The medical device may comprise a delivery system carrying a heart valve repair device or another medical device for a procedure in or around a heart valve. The heart valve repair device may comprise a ventricular winding having a generally spiral shape adapted to be positioned on a ventricular side of the heart valve such that chords associated with the heart valve are positioned within the path of the generally spiral shape of the ventricular winding. The method may further comprise the step of advancing distally an assembly comprising the steerable introducer and the delivery system carrying the heart valve repair device, after the step of adjusting the distal tip of the flexible tubular catheter to face the tricuspid valve, and before the step of operating the steerable introducer to move the distal tip toward the desired valve area. The step of advancing distally the assembly comprising the steerable introducer and the delivery system carrying the heart valve repair device may comprise advancing the ventricular winding into the right ventricle. The step of advancing distally the assembly comprising the steerable introducer and the delivery system carrying the heart valve repair device may comprise advancing the assembly about 3 centimeters distally.

The step of operating the steerable introducer to move the distal tip toward a desired valve area may comprise moving the distal tip toward an area of the tricuspid valve commissure between the anterior leaflet and the posterior leaflet of the tricuspid valve, and may further comprise subsequently operating the steerable introducer in a manner selected from the group of: (i)(a) to move the distal tip toward the anterior leaflet of the tricuspid valve, turning the control knob in a direction of straightening the distal tip of the flexible tubular catheter, for example by about one-eighth turn of the control knob, and (i)(b) to move the distal tip toward the posterior leaflet of the tricuspid valve, turning the control knob in a direction of bending the distal tip of the flexible tubular catheter, for example by about one-eighth turn of the control knob.

The step of operating the steerable introducer to move the distal tip toward a desired valve area may comprise moving the distal tip toward an area of the tricuspid valve commissure between the posterior leaflet and the septal leaflet of the tricuspid valve, and may further comprise subsequently operating the steerable introducer in a manner selected from the group of: (ii)(a) to move the distal tip toward the posterior leaflet of the tricuspid valve, turning the control knob in a direction of bending the distal tip of the flexible tubular catheter, for example by about one-eighth turn of the control knob, and rotating the flexible tubular catheter around its longitudinal axis in a counterclockwise direction when viewed distally, for example by about one-eighth turn, and (ii)(b) to move the distal tip toward the septal leaflet of the tricuspid valve, turning the control knob in a direction of straightening the distal tip of the flexible tubular catheter, for example by about one-eighth turn of the control knob, and rotating the flexible tubular catheter around its longitudinal axis in a clockwise direction when viewed distally, for example by about one-eighth turn.

The step of operating the steerable introducer to move the distal tip toward a desired valve area may comprise moving the distal tip toward an area of the tricuspid valve commissure between the septal leaflet and the anterior leaflet of the tricuspid valve, and may further comprise subsequently operating the steerable introducer in a manner selected from the group of: (iii)(a) to move the distal tip toward the septal leaflet of the tricuspid valve, rotating the flexible tubular catheter around its longitudinal axis in a clockwise direction when viewed distally, for example by about one-eighth turn, and (iii)(b) to move the distal tip toward the anterior leaflet of the tricuspid valve, rotating the flexible tubular catheter around its longitudinal axis in a counterclockwise direction when viewed distally, for example by about one-eighth turn.

Another example method for positioning a medical device relative to heart valve anatomy of a patient comprises the steps of: inserting a steerable introducer into the patient, the steerable introducer comprising (a) a flexible tubular catheter comprising a longitudinal axis and distal tip and (b) a control knob for bending and unbending the distal tip; inserting a medical device through the steerable introducer; moving the distal tip of the flexible tubular catheter into approximately a center of the left atrium of the heart of the patient; adjusting the distal tip of the flexible tubular catheter to face the mitral valve of the patient between the left atrium and the left ventricle; and operating the steerable introducer to move the distal tip toward a desired valve area in a manner selected from the group of: (i) to move the distal tip toward an area of the mitral valve commissure on a side of the anterior papillary muscle, turning the control knob in a direction of straightening the distal tip of the flexible tubular catheter, for example by about one-quarter turn of the control knob, and (ii) to move the distal tip toward an area of the mitral valve commissure on a side of the posterior papillary muscle, turning the control knob in a direction of bending the distal tip of the flexible tubular catheter, for example by about one-quarter turn of the control knob.

The step of adjusting the distal tip of the flexible tubular catheter to face the mitral valve comprises turning the control knob in a direction of bending the distal tip of the flexible tubular catheter, for example by about one-quarter turn to about one-half turn of the control knob.

The step of operating the steerable introducer to move the distal tip toward a desired valve area may comprise moving the distal tip toward an area of the mitral valve commissure on a side of the anterior papillary muscle, and may further comprise subsequently operating the steerable introducer in a manner selected from the group of: (i)(a) to move the distal tip toward the anterior leaflet of the mitral valve, rotating the flexible tubular catheter around its longitudinal axis in a counterclockwise direction when viewed distally, for example by about one-eighth turn, and (i)(b) to move the distal tip toward the posterior leaflet of the mitral valve, rotating the flexible tubular catheter around its longitudinal axis in a clockwise direction when viewed distally, for example by about one-eighth turn.

The step of operating the steerable introducer to move the distal tip toward a desired valve area may comprise moving the distal tip toward an area of the mitral valve commissure on a side of the posterior papillary muscle, and may further comprise subsequently operating the steerable introducer in a manner selected from the group of: (ii)(a) to move the distal tip toward the anterior leaflet of the mitral valve, rotating the flexible tubular catheter around its longitudinal axis in a counterclockwise direction when viewed distally, for example by about one-eighth turn, and (ii)(b) to move the distal tip toward the posterior leaflet of the mitral valve, rotating the flexible tubular catheter around its longitudinal axis in a clockwise direction when viewed distally, for example by about one-eighth turn.

Example methods include following a road map of steps as described herein to a desired position relative to a tricuspid valve or a mitral valve.

### BRIEF DESCRIPTION OF THE DRAWINGS

FIG. 1 shows a steerable introducer as known in the art.

FIG. 2 shows an assembly comprising the steerable introducer of FIG. 1 with a medical device comprising a delivery system and a stand to which the steerable introducer and medical device are mounted.

FIG. 3 shows a cross-section of a heart with the distal tip of a steerable introducer located in the right atrium.

FIG. 4 shows a cross-section of a heart with the position of the distal tip of the steerable introducer adjusted to face the tricuspid valve.

FIG. 5 shows a cross-section of a heart with a heart valve repair device advanced distally into the right ventricle.

FIG. 6 shows a cross-section of a heart with the distal tip of a steerable introducer located in the right atrium by an alternate approach.

FIG. 7 shows a cross-section of a heart with the position of the distal tip of the steerable introducer adjusted to face the tricuspid valve by an alternate approach.

FIG. 8 shows a cross-section of a heart with a heart valve repair device advanced distally into the right ventricle by an alternate approach.

FIG. 9 is a flow chart showing steps in moving a medical device into a ventricle.

FIG. 10 is a flow chart map showing steps for navigating from a central position to different positions relative to a tricuspid valve.

FIG. 11 is a flow chart map showing steps for navigating from a central position to different positions relative to a mitral valve.

### DETAILED DESCRIPTION

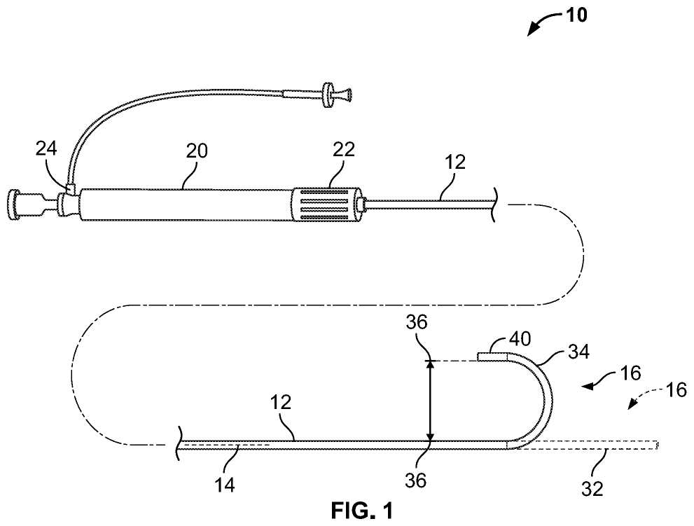

FIG. 1 shows a steerable introducer as known in the art. One example of a steerable introducer suitable for use with the invention is the AGILIS NxT Steerable Introducer of St. Jude Medical, Inc. (subsidiary of Abbott). For example, the steerable introducer may be the AGILIS NxT Steerable Introducer w/Tip Marker, size 8.5 French, 91 centimeter lumen length, 71 centimeter usable length, with straightenable 3 millimeter bi-directional “J” distal tip (16.8 millimeter, 22.4 millimeter, or 50.0 millimeter curl).

Other sizes are available and usable with the methods described herein, for example size 8.5 French, 81 or 102 centimeter lumen length, 61 or 82 centimeter usable length, with straightenable 3 millimeter bi-directional “J” distal tip (16.8 millimeter, 22.4 millimeter, or 50.0 millimeter curl). These are just examples, as other sizes may be used.

As shown in FIG. 1, the steerable introducer 10 comprises a flexible tubular catheter 12 comprising a longitudinal axis 14 (extending along the length of the flexible tubular catheter 12, illustrated as a dashed line along a portion of the length in FIG. 1) and distal tip 16. The steerable introducer further comprises a handle 20 and a control knob 22 that can be rotated about an axis. By rotating the control knob 22 relative to the handle 20, an operator can cause the distal tip 16 to move in a controlled fashion between a straight position 32 (shown in phantom lines in FIG. 1) and a fully bent “J” position 34 (as shown in FIG. 1), as well as to positions in between. When fully bent, the curl dimension 36 may be, for example, 16.8 millimeter, 22.4 millimeter, or 50.0 millimeter. The operator also may rotate the flexible tubular catheter 12 around its longitudinal axis 14 in a clockwise or counterclockwise direction (when viewed distally) by rotating the handle 20. The steerable introducer 10 may include a tip marker 40 for visualization of the location of the end of the distal tip 16 during a procedure. The steerable introducer 10 may also include an irrigation port 24 as shown in FIG. 1.

The steerable introducer 10 may be used, for example, in positioning a heart valve repair device. As one example, the steerable introducer 10 may be used with a heart valve repair device and delivery system as disclosed in U.S. Pat. No. 9,700,412, the contents of which are hereby incorporated by reference herein. For example, the steerable introducer 10 may be used with a heart valve repair device and delivery system as shown in FIGS. 6, 7A, 7B, 8, and 9 of U.S. Pat. No. 9,700,412, incorporated by reference herein. As disclosed in that patent, the heart valve repair device may comprise a ventricular winding having a generally spiral shape adapted to be positioned on a ventricular side of the heart valve such that chords associated with the heart valve are positioned within the path of the generally spiral shape of the ventricular winding.

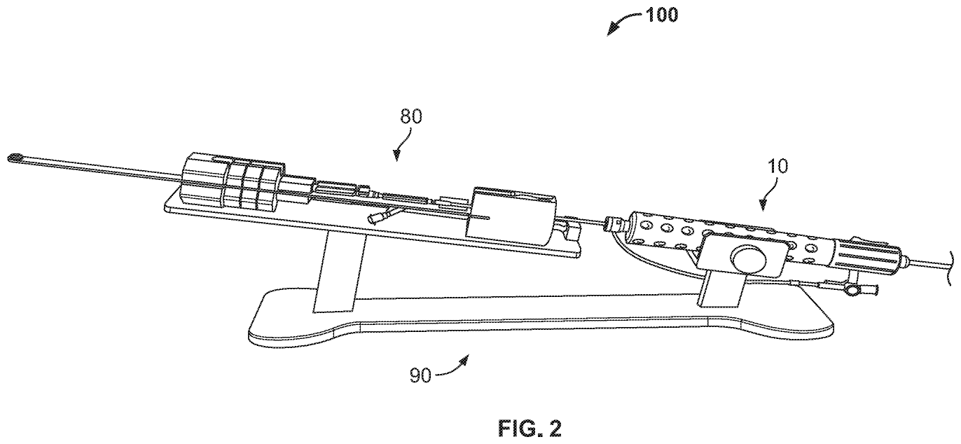

FIG. 2 shows an assembly 100 comprising the steerable introducer 10 of FIG. 1 with a medical device comprising a delivery system 80. The assembly 100 further comprises a stand 90 to which the steerable introducer 10 and delivery system 80 of the medical device are mounted.

In a starting position for example procedures as illustrated, the steerable introducer 10 and delivery system 80 are mounted to the stand 90. The irrigation port 24 is oriented to face away from the operator, parallel to the table on which the stand 90 rests. In other words, for the starting position, when facing distally down the axis of the steerable introducer 10 toward the patient, and considering the position of the irrigation port 24 as on a clock face, the irrigation port 24 is oriented to face the 3:00 position, or approximately the 3:00 position.

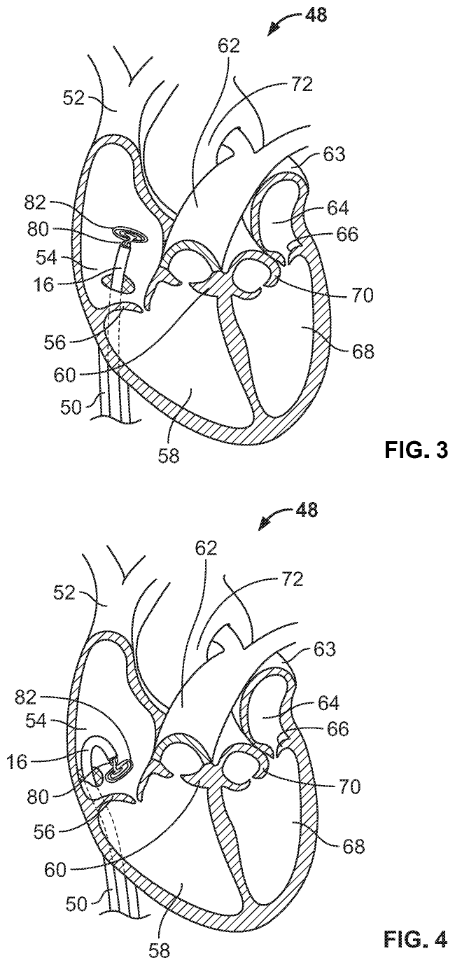

FIG. 3 shows a cross-section of a heart 48 with the distal tip 16 of a steerable introducer 10 located in the right atrium 54. The heart anatomy illustrated includes inferior vena cava 50, superior vena cava 52, right atrium 54, tricuspid valve 56, right ventricle 58, pulmonary valve 60, pulmonary artery 62, pulmonary vein 63, left atrium 64, mitral valve 66, right ventricle 68, aortic valve 70, and aorta 72.

As shown in FIG. 3, the distal tip 16 of a steerable introducer 10 has been moved into the right atrium 54 via the inferior vena cava 50. The steerable introducer 10 has been previously inserted into the patient, for example via a femoral vein, and tracked through the vasculature to the inferior vena cava 50 and into the heart 48, although other approaches are possible. In this example, a medical device is shown inserted through the steerable introducer 10. The medical device includes a delivery system 80 and a heart valve repair device 82. The heart valve repair device 82 and delivery system 80 may be similar to the heart valve repair device 110 and delivery system 300 as shown in FIGS. 6, 7A, 7B, 8, and 9 of U.S. Pat. No. 9,700,412, incorporated by reference herein. The operator may insert the medical device (e.g., delivery system 300 carrying heart valve repair device 110) through the steerable introducer 10 either before or after the step of inserting the steerable introducer 10 into the patient.

In this example, for deployment of the heart valve repair device 110, the operator moves the distal tip 16 of the flexible tubular catheter 12 of the steerable introducer 10 into approximately a center of the right atrium 54 of the heart 48 of the patient, as shown in FIG. 3. Then, the operator adjusts the distal tip 16 of the flexible tubular catheter 12 of the steerable introducer 10 to face the tricuspid valve 56 of the patient between the right atrium 54 and the right ventricle 58, as shown in FIG. 4. In order to perform this adjustment, the operator turns the control knob 22 in a direction of bending the distal tip 16 of the flexible tubular catheter 12 of the steerable introducer 10, for example by about one-quarter turn to about one-half turn. FIG. 4 shows the steerable introducer 10 after the operator has adjusted the distal tip 16 of the flexible tubular catheter 12 of the steerable introducer 10 to face the tricuspid valve 56.

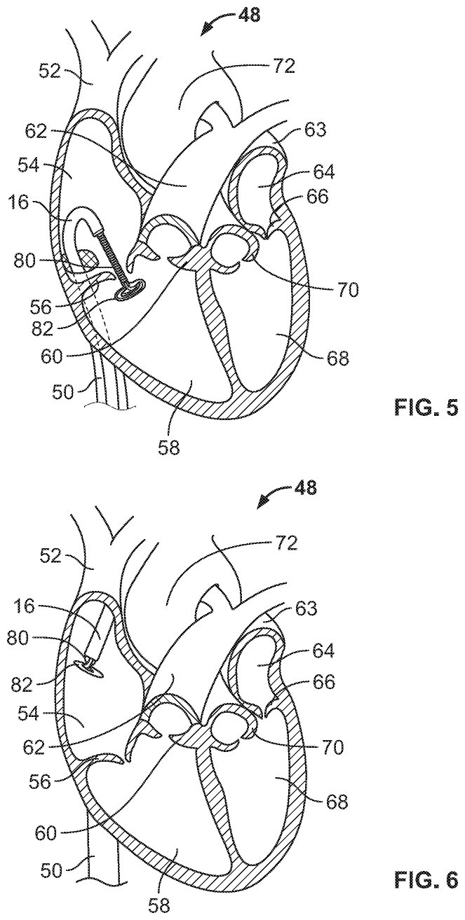

In this example, once the operator has adjusted the distal tip 16 of the flexible tubular catheter 12 of the steerable introducer 10 to face the tricuspid valve 56, the operator may advance the assembly 100 comprising the stand 90, the steerable introducer 10, and the delivery system 80 carrying the heart valve repair device 82 distally (i.e., toward the patient). The step of advancing the assembly 100 distally may comprise advancing the ventricular winding of the heart valve repair device 82 into the right ventricle 58. In this example, the step of advancing the assembly 100 distally comprises advancing the assembly 100 about 3 centimeters distally. FIG. 5 shows the steerable introducer 10 and heart valve repair device 82 after the operator has advanced the assembly 100 distally. The advancement of the assembly 100 may result in the heart valve repair device 82 extending further from the distal tip 16 of the flexible tubular catheter 12. As shown in FIG. 5, the heart valve repair device 82 has been advanced through the tricuspid valve 56 into the right ventricle 58. The advancement of the heart valve repair device 82 into the ventricle may be accomplished by the step of advancing the assembly 100 and/or by a separate subsequent step of advancing the delivery system.

FIGS. 6-8 are similar to FIGS. 3-5 but illustrate an alternate approach into the right atrium, via the superior vena cava 52 instead of the inferior vena cava 50. In this approach, the same starting position for the assembly 100 as described above may be used, with the irrigation port 24 oriented to face the 3:00 position, or approximately the 3:00 position, when facing distally down the axis of the steerable introducer 10 toward the patient.

FIG. 6 shows a cross-section of a heart 48 with the distal tip 16 of a steerable introducer 10 located in the right atrium 54. As shown in FIG. 6, the distal tip 16 of a steerable introducer 10 has been moved into the right atrium 54 via the superior vena cava 52. The steerable introducer has been previously inserted into the patient, for example via a femoral vein, and tracked through the vasculature to the superior vena cava 52 and into the heart 48.

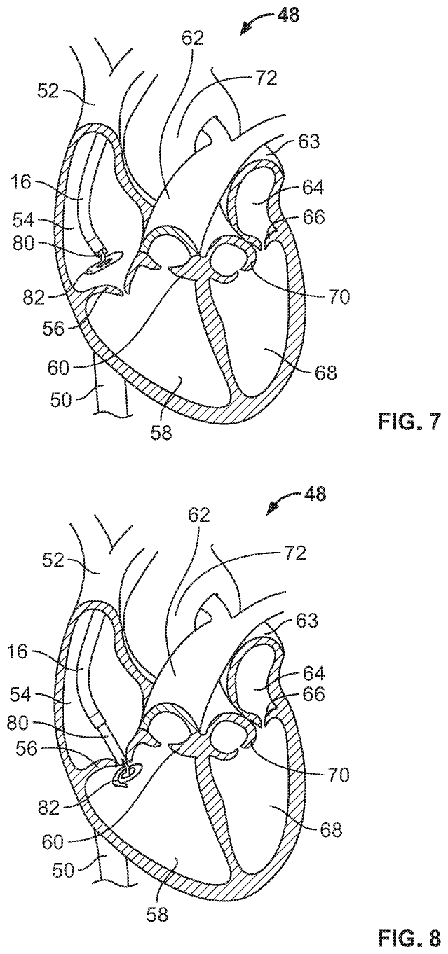

As with the previous example, in this example, for deployment of the heart valve repair device 110, the operator moves the distal tip 16 of the flexible tubular catheter 12 of the steerable introducer 10 into approximately a center of the right atrium 54 of the heart 48 of the patient. Then, the operator adjusts the distal tip 16 of the flexible tubular catheter 12 of the steerable introducer 10 to face the tricuspid valve 56 of the patient between the right atrium 54 and the right ventricle 58, as shown in FIG. 7. In order to perform this adjustment, the operator turns the control knob 22 in a direction of bending the distal tip 16 of the flexible tubular catheter 12 of the steerable introducer 10, for example by about one-quarter turn to about one-half turn. FIG. 7 shows the steerable introducer 10 after the operator has adjusted the distal tip 16 of the flexible tubular catheter 12 of the steerable introducer 10 to face the tricuspid valve 56.

As with the previous example, in this example, once the operator has adjusted the distal tip 16 of the flexible tubular catheter 12 of the steerable introducer 10 to face the tricuspid valve 56, the operator may advance the assembly 100 comprising the stand 90, the steerable introducer 10, and the delivery system 80 carrying the heart valve repair device 82 distally (i.e., toward the patient), for example by about 3 centimeters distally. FIG. 8 shows the steerable introducer 10 and heart valve repair device 82 after the operator has advanced the assembly 100 distally. The advancement of the assembly 100 may result in the heart valve repair device 82 extending further from the distal tip 16 of the flexible tubular catheter 12. As shown in FIG. 8, the heart valve repair device 82 has been advanced through the tricuspid valve 56 into the right ventricle 58. The advancement of the heart valve repair device 82 into the ventricle may be accomplished by the step of advancing the assembly 100 and/or by a separate subsequent step of advancing the delivery system.

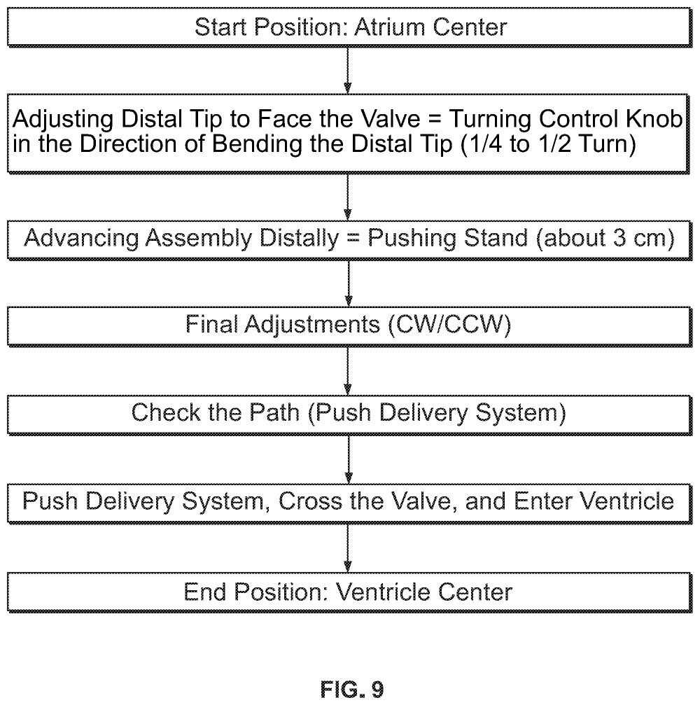

FIG. 9 is a flow chart showing steps in moving a medical device into a ventricle. For the steps in this flow chart, the same starting position for the assembly 100 as described above may be used, with the irrigation port 24 oriented to face the 3:00 position, or approximately the 3:00 position, when facing distally down the axis of the steerable introducer 10 toward the patient. To get to the start position for this flow chart, the operator moves the distal tip of the steerable introducer into approximately a center of an atrium of the heart of the patient. Then, the operator adjusts the distal tip of the steerable introducer to face the valve of the patient between the atrium and the ventricle. In order to perform this adjustment, the operator turns the control knob of the steerable introducer in a direction of bending the distal tip, for example by about one-quarter turn to about one-half turn. Once the operator has adjusted the distal tip of the steerable introducer to face the valve, the operator may advance the assembly distally, for example by pushing the stand 90 distally (toward the patient). The step of advancing the assembly distally may comprise advancing the stand by about 3 centimeters distally. The advancement of the heart valve repair device into the ventricle may be accomplished by this step of advancing the assembly and/or by a separate subsequent step of advancing the delivery system. After advancing the assembly distally, further adjustments may be made to position the medical device in the central position. For example, the operator may rotate the flexible tubular catheter around its longitudinal axis in a clockwise or counterclockwise direction. Then, the operator may check the path by pushing the delivery system distally. A subsequent step may be added of pushing the delivery system distally if needed for the device to cross the valve and enter the ventricle. The desired end position is centrally with respect to the ventricle in a radial direction.

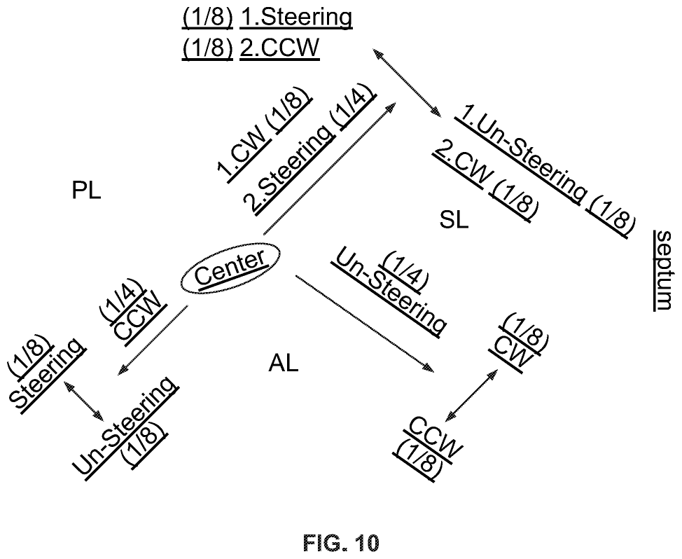

FIG. 10 is a flow chart map showing steps for navigating from a central starting position to different positions relative to a tricuspid valve. The central position for this flow chart is the position in which the medical device is positioned in a central location relative to the tricuspid valve 56, such as just below the center of the tricuspid valve 56 in the right ventricle 58. This position may be the position reached after the steps of FIG. 9. From this starting position, FIG. 10 illustrates a map of the tricuspid valve and the steps to move from the central starting position in various directions toward the commissure of the valve and around the periphery of the valve.

Thus, as shown in FIG. 10, the operator may operate the steerable introducer to move the distal tip toward a desired valve area in a manner selected from the group of: (i) to move the distal tip toward an area of the tricuspid valve commissure between the anterior leaflet (AL) and the posterior leaflet (PL) of the tricuspid valve, rotating the flexible tubular catheter around its longitudinal axis in a counterclockwise direction when viewed distally, for example by about one-quarter turn, (ii) to move the distal tip toward an area of the tricuspid valve commissure between the posterior leaflet (PL) and the septal leaflet (SL) of the tricuspid valve, rotating the flexible tubular catheter around its longitudinal axis in a clockwise direction when viewed distally, for example by about one-eighth turn, and turning the control knob in a direction of bending the distal tip of the flexible tubular catheter for example by about one-quarter turn of the control knob, and (iii) to move the distal tip toward an area of the tricuspid valve commissure between the septal leaflet (SL) and the anterior leaflet (AL) of the tricuspid valve, turning the control knob in a direction of straightening the distal tip of the flexible tubular catheter, for example by about one-quarter turn of the control knob.

If the operator moves the distal tip toward an area of the tricuspid valve commissure between the anterior leaflet and the posterior leaflet of the tricuspid valve, the operator may subsequently operate the steerable introducer to move the distal tip around a periphery of the valve in a manner selected from the group of: (i)(a) to move the distal tip toward the anterior leaflet of the tricuspid valve, turning the control knob in a direction of straightening the distal tip of the flexible tubular catheter, for example by about one-eighth turn of the control knob, and (i)(b) to move the distal tip toward the posterior leaflet of the tricuspid valve, turning the control knob in a direction of bending the distal tip of the flexible tubular catheter, for example by about one-eighth turn of the control knob.

If the operator moves the distal tip toward an area of the tricuspid valve commissure between the posterior leaflet and the septal leaflet of the tricuspid valve, the operator may subsequently operate the steerable introducer to move the distal tip around a periphery of the valve in a manner selected from the group of: (ii)(a) to move the distal tip toward the posterior leaflet of the tricuspid valve, turning the control knob in a direction of bending the distal tip of the flexible tubular catheter, for example by about one-eighth turn of the control knob, and rotating the flexible tubular catheter around its longitudinal axis in a counterclockwise direction when viewed distally, for example by about one-eighth turn, and (ii)(b) to move the distal tip toward the septal leaflet of the tricuspid valve, turning the control knob in a direction of straightening the distal tip of the flexible tubular catheter, for example by about one-eighth turn of the control knob, and rotating the flexible tubular catheter around its longitudinal axis in a clockwise direction when viewed distally, for example by about one-eighth turn.

If the operator moves the distal tip toward an area of the tricuspid valve commissure between the septal leaflet and the anterior leaflet of the tricuspid valve, the operator may subsequently operate the steerable introducer to move the distal tip around a periphery of the valve in a manner selected from the group of: (iii)(a) to move the distal tip toward the septal leaflet of the tricuspid valve, rotating the flexible tubular catheter around its longitudinal axis in a clockwise direction when viewed distally, for example by about one-eighth turn, and (iii)(b) to move the distal tip toward the anterior leaflet of the tricuspid valve, rotating the flexible tubular catheter around its longitudinal axis in a counterclockwise direction when viewed distally, for example by about one-eighth turn.

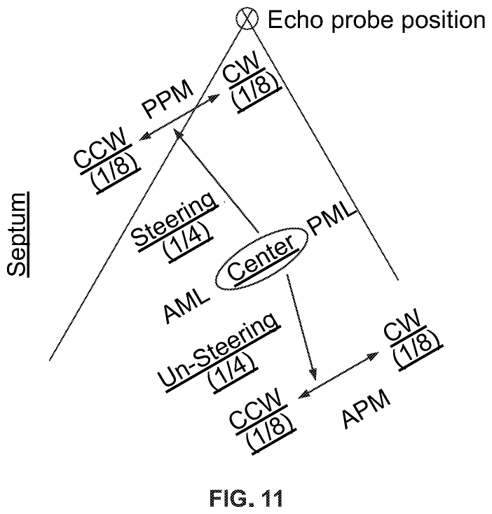

FIG. 11 is a flow chart map showing steps for navigating from a central position to different positions relative to a mitral valve. The left atrium 64 may be reached by any suitable approach, such as one of the approaches described above for reaching the right atrium 54 and then crossing the septum into the left atrium 64. The central position for this flow chart is the position in which the medical device is positioned in a central location relative to the mitral valve 66, such as just below the center of the mitral valve 66 in the left ventricle 68. From this central starting position, FIG. 11 illustrates a map of the mitral valve and the steps to move from the central starting position in various directions toward the commissure of the valve and around the periphery of the valve.

Thus, as shown in FIG. 11, the operator may operate the steerable introducer to move the distal tip toward a desired valve area in a manner selected from the group of: (i) to move the distal tip toward an area of the mitral valve commissure on a side of the anterior papillary muscle (APM), turning the control knob in a direction of straightening the distal tip of the flexible tubular catheter, for example by about one-quarter turn of the control knob, and (ii) to move the distal tip toward an area of the mitral valve commissure on a side of the posterior papillary muscle (PPM), turning the control knob in a direction of bending the distal tip of the flexible tubular catheter, for example by about one-quarter turn of the control knob.

If the operator moves the distal tip toward an area of the mitral valve commissure on a side of the anterior papillary muscle, the operator may subsequently operate the steerable introducer to move the distal tip around a periphery of the valve in a manner selected from the group of: (i)(a) to move the distal tip toward the anterior leaflet (AML) of the mitral valve, rotating the flexible tubular catheter around its longitudinal axis in a counterclockwise direction when viewed distally, for example by about one-eighth turn, and (i)(b) to move the distal tip toward the posterior leaflet (PML) of the mitral valve, rotating the flexible tubular catheter around its longitudinal axis in a clockwise direction when viewed distally, for example by about one-eighth turn.

If the operator moves the distal tip toward an area of the mitral valve commissure on a side of the posterior papillary muscle, the operator may subsequently operate the steerable introducer to move the distal tip around a periphery of the valve in a manner selected from the group of: (ii)(a) to move the distal tip toward the anterior leaflet (AML) of the mitral valve, rotating the flexible tubular catheter around its longitudinal axis in a counterclockwise direction when viewed distally, for example by about one-eighth turn, and (ii)(b) to move the distal tip toward the posterior leaflet (PML) of the mitral valve, rotating the flexible tubular catheter around its longitudinal axis in a clockwise direction when viewed distally, for example by about one-eighth turn.

The steps as described herein to reach a location may be followed in reverse to reach a different location. For example, if the device is located near the commissure, the steps may be followed in reverse to bring the device to a central position, and then, if desired, the steps may be followed to a different position located near the commissure.

The above description and accompanying illustrations provide useful road maps with predefined steps for positioning a steerable introducer and medical device relative to a heart valve. By having these road maps and these predefined steps available, the operator knows which steps to perform in order to position to the desired location. The operator also may follow the steps in the road maps in a backwards direction to move to a different position, such as to get from a position toward or around the commissure to a central position and then to advance to a different position toward or around the commissure.

Selecting from these sets of predefined steps provides advantageous improvements over the prior methods of steering. In particular, the invention provides improved systems and methods for positioning medical devices relative to heart valve anatomy that reduce procedure time and risk, reduce cost, improve ease of use, and/or improve overall clinical outcome.

The description herein uses various dimensions for turning the control knob of the steerable introducer, for rotating the steerable introducer, and for advancing the assembly. Certain dimensions above use the term “about” which would be understood to include the dimension stated as well as more or less depending upon the circumstances of the procedure, for example depending upon the particular patient. The terms “distal” and “proximal” have meanings as generally understood in the art: “distal” movement refers to advancement away from the operator and toward the patient; “proximal” movement refers to retraction toward the operator. The Figures accompanying this description are for illustrative purposes only and are not to scale.

The embodiments described and illustrated herein are only examples, as many variations are possible. The materials, dimensions, components, order of steps, and operation may be varied without departing from the scope of the invention, which is limited only by the appended claims.

What is claimed is:

1. A method for positioning a medical device relative to heart valve anatomy of a patient, comprising the steps of:
inserting a steerable introducer into the patient, the steerable introducer comprising (a) a flexible tubular catheter comprising a longitudinal axis and distal tip and (b) a control knob for bending and straightening the distal tip;
inserting a medical device through the steerable introducer;
moving the distal tip of the flexible tubular catheter into approximately a center of the right atrium of the heart of the patient;
adjusting the distal tip of the flexible tubular catheter to face the tricuspid valve of the patient between the right atrium and the right ventricle; and
operating the steerable introducer to move the distal tip toward a desired valve area in a manner selected from the group of: (i) to move the distal tip toward an area of the tricuspid valve commissure between the anterior leaflet and the posterior leaflet of the tricuspid valve, rotating the flexible tubular catheter around its longitudinal axis in a counterclockwise direction when viewed distally, (ii) to move the distal tip toward an area of the tricuspid valve commissure between the posterior leaflet and the septal leaflet of the tricuspid valve, rotating the flexible tubular catheter around its longitudinal axis in a clockwise direction when viewed distally, and turning the control knob in a direction of bending the distal tip of the flexible tubular catheter, and (iii) to move the distal tip toward an area of the tricuspid valve commissure between the septal leaflet and the anterior leaflet of the tricuspid valve, turning the control knob in a direction of straightening the distal tip of the flexible tubular catheter.

2. A method as in claim 1, wherein the step of operating the steerable introducer to move the distal tip toward a desired valve area comprises operating the steerable introducer in a manner selected from the group of: (i) to move the distal tip toward an area of the tricuspid valve commissure between the anterior leaflet and the posterior leaflet of the tricuspid valve, rotating the flexible tubular catheter around its longitudinal axis by about one-quarter turn in a counterclockwise direction when viewed distally, (ii) to move the distal tip toward an area of the tricuspid valve commissure between the posterior leaflet and the septal leaflet of the tricuspid valve, rotating the flexible tubular catheter around its longitudinal axis by about one-eighth turn in a clockwise direction when viewed distally, and turning the control knob by about one-quarter turn in a direction of bending the distal tip of the flexible tubular catheter, and (iii) to move the distal tip toward an area of the tricuspid valve commissure between the septal leaflet and the anterior leaflet of the tricuspid valve, turning the control knob by about one-quarter turn in a direction of straightening the distal tip of the flexible tubular catheter.

3. A method as in claim 1, wherein the step of adjusting the distal tip of the flexible tubular catheter to face the tricuspid valve comprises turning the control knob in a direction of bending the distal tip of the flexible tubular catheter.

4. A method as in claim 1, wherein the medical device comprises a delivery system carrying a heart valve repair device, wherein the heart valve repair device comprises a ventricular winding having a generally spiral shape adapted to be positioned on a ventricular side of the heart valve such that chords associated with the heart valve are positioned within the path of the generally spiral shape of the ventricular winding, and further comprising the step of advancing distally an assembly comprising the steerable introducer and the delivery system carrying the heart valve repair device, after the step of adjusting the distal tip of the flexible tubular catheter to face the tricuspid valve, and before the step of operating the steerable introducer to move the distal tip toward the desired valve area.

5. A method as in claim 4, wherein the step of advancing distally the assembly comprising the steerable introducer and the delivery system carrying the heart valve repair device comprises advancing the ventricular winding into the right ventricle.

6. A method as in claim 1, wherein the step of operating the steerable introducer to move the distal tip toward a desired valve area comprises moving the distal tip toward an area of the tricuspid valve commissure between the anterior leaflet and the posterior leaflet of the tricuspid valve, and further comprises subsequently operating the steerable introducer in a manner selected from the group of: (i)(a) to move the distal tip toward the anterior leaflet of the tricuspid valve, turning the control knob in a direction of straightening the distal tip of the flexible tubular catheter, and (i)(b) to move the distal tip toward the posterior leaflet of the tricuspid valve, turning the control knob in a direction of bending the distal tip of the flexible tubular catheter.

7. A method as in claim 1, wherein the step of operating the steerable introducer to move the distal tip toward a desired valve area comprises moving the distal tip toward an area of the tricuspid valve commissure between the anterior leaflet and the posterior leaflet of the tricuspid valve, and further comprises subsequently operating the steerable introducer in a manner selected from the group of: (i)(a) to move the distal tip toward the anterior leaflet of the tricuspid valve, turning the control knob by about one-eighth turn in a direction of straightening the distal tip of the flexible tubular catheter, and (i)(b) to move the distal tip toward the posterior leaflet of the tricuspid valve, turning the control knob by about one-eighth turn in a direction of bending the distal tip of the flexible tubular catheter.

8. A method as in claim 1, wherein the step of operating the steerable introducer to move the distal tip toward a desired valve area comprises moving the distal tip toward an area of the tricuspid valve commissure between the posterior leaflet and the septal leaflet of the tricuspid valve, and further comprises subsequently operating the steerable introducer in a manner selected from the group of: (ii)(a) to move the distal tip toward the posterior leaflet of the tricuspid valve, turning the control knob in a direction of bending the distal tip of the flexible tubular catheter, and rotating the flexible tubular catheter around its longitudinal axis in a counterclockwise direction when viewed distally, and (ii)(b) to move the distal tip toward the septal leaflet of the tricuspid valve, turning the control knob in a direction of straightening the distal tip of the flexible tubular catheter, and rotating the flexible tubular catheter around its longitudinal axis in a clockwise direction when viewed distally.

9. A method as in claim 1, wherein the step of operating the steerable introducer to move the distal tip toward a desired valve area comprises moving the distal tip toward an area of the tricuspid valve commissure between the posterior leaflet and the septal leaflet of the tricuspid valve, and further comprises subsequently operating the steerable introducer in a manner selected from the group of: (ii)(a) to move the distal tip toward the posterior leaflet of the tricuspid valve, turning the control knob by about one-eighth turn in a direction of bending the distal tip of the flexible tubular catheter, and rotating the flexible tubular catheter around its longitudinal axis by about one-eighth turn in a counterclockwise direction when viewed distally, and (ii)(b) to move the distal tip toward the septal leaflet of the tricuspid valve, turning the control knob by about one-eighth turn in a direction of straightening the distal tip of the flexible tubular catheter, and rotating the flexible tubular catheter around its longitudinal axis by about one-eighth turn in a clockwise direction when viewed distally.

10. A method as in claim 1, wherein the step of operating the steerable introducer to move the distal tip toward a desired valve area comprises moving the distal tip toward an area of the tricuspid valve commissure between the septal leaflet and the anterior leaflet of the tricuspid valve, and further comprises subsequently operating the steerable introducer in a manner selected from the group of: (iii)(a) to move the distal tip toward the septal leaflet of the tricuspid valve, rotating the flexible tubular catheter around its longitudinal axis in a clockwise direction when viewed distally, and (iii)(b) to move the distal tip toward the anterior leaflet of the tricuspid valve, rotating the flexible tubular catheter around its longitudinal axis in a counterclockwise direction when viewed distally.

11. A method as in claim 1, wherein the step of operating the steerable introducer to move the distal tip toward a desired valve area comprises moving the distal tip toward an area of the tricuspid valve commissure between the septal leaflet and the anterior leaflet of the tricuspid valve, and further comprises subsequently operating the steerable introducer in a manner selected from the group of: (iii)(a) to move the distal tip toward the septal leaflet of the tricuspid valve, rotating the flexible tubular catheter around its longitudinal axis by about one-eighth turn in a clockwise direction when viewed distally, and (iii)(b) to move the distal tip toward the anterior leaflet of the tricuspid valve, rotating the flexible tubular catheter around its longitudinal axis by about one-eighth turn in a counterclockwise direction when viewed distally.

12. A method for positioning a medical device relative to heart valve anatomy of a patient, comprising the steps of:
inserting a steerable introducer into the patient, the steerable introducer comprising (a) a flexible tubular catheter comprising a longitudinal axis and distal tip and (b) a control knob for bending and unbending the distal tip;
inserting a medical device through the steerable introducer;
moving the distal tip of the flexible tubular catheter into approximately a center of the left atrium of the heart of the patient;
adjusting the distal tip of the flexible tubular catheter to face the mitral valve of the patient between the left atrium and the left ventricle; and
operating the steerable introducer to move the distal tip toward a desired valve area in a manner selected from the group of: (i) to move the distal tip toward an area of the mitral valve commissure on a side of the anterior papillary muscle, turning the control knob in a direction of straightening the distal tip of the flexible tubular catheter, and (ii) to move the distal tip toward an area of the mitral valve commissure on a side of the posterior papillary muscle, turning the control knob in a direction of bending the distal tip of the flexible tubular catheter.

13. A method as in claim 12, wherein the step of operating the steerable introducer to move the distal tip toward a desired valve area comprises operating the steerable introducer in a manner selected from the group of: (i) to move the distal tip toward an area of the mitral valve commissure on a side of the anterior papillary muscle, turning the control knob by about one-quarter turn in a direction of straightening the distal tip of the flexible tubular catheter, and (ii) to move the distal tip toward an area of the mitral valve commissure on a side of the posterior papillary muscle, turning the control knob by about one-quarter turn in a direction of bending the distal tip of the flexible tubular catheter.

14. A method as in claim 12, wherein the step of adjusting the distal tip of the flexible tubular catheter to face the mitral valve comprises turning the control knob in a direction of bending the distal tip of the flexible tubular catheter.

15. A method as in claim 12, wherein the medical device comprises a delivery system carrying a heart valve repair device, wherein the heart valve repair device comprises a ventricular winding having a generally spiral shape adapted to be positioned on a ventricular side of the heart valve such that chords associated with the heart valve are positioned within the path of the generally spiral shape of the ventricular winding, and further comprising the step of advancing distally an assembly comprising the steerable introducer and the delivery system carrying the heart valve repair device, after the step of adjusting the distal tip of the flexible tubular catheter to face the mitral valve, and before the step of operating the steerable introducer to move the distal tip toward the desired valve area.

16. A method as in claim 15, wherein the step of advancing distally the assembly comprising the steerable introducer and the delivery system carrying the heart valve repair device comprises advancing the ventricular winding into the left ventricle.

17. A method as in claim 12, wherein the step of operating the steerable introducer to move the distal tip toward a desired valve area comprises moving the distal tip toward an area of the mitral valve commissure on a side of the anterior papillary muscle, and further comprises subsequently operating the steerable introducer in a manner selected from the group of: (i)(a) to move the distal tip toward the anterior leaflet of the mitral valve, rotating the flexible tubular catheter around its longitudinal axis in a counterclockwise direction when viewed distally, and (i)(b) to move the distal tip toward the posterior leaflet of the mitral valve, rotating the flexible tubular catheter around its longitudinal axis in a clockwise direction when viewed distally.

18. A method as in claim 12, wherein the step of operating the steerable introducer to move the distal tip toward a desired valve area comprises moving the distal tip toward an area of the mitral valve commissure on a side of the anterior papillary muscle, and further comprises subsequently operating the steerable introducer in a manner selected from the group of: (i)(a) to move the distal tip toward the anterior leaflet of the mitral valve, rotating the flexible tubular catheter around its longitudinal axis by about one-eighth turn in a counterclockwise direction when viewed distally, and (i)(b) to move the distal tip toward the posterior leaflet of the mitral valve, rotating the flexible tubular catheter around its longitudinal axis by about one-eighth turn in a clockwise direction when viewed distally.

19. A method as in claim 12, wherein the step of operating the steerable introducer to move the distal tip toward a desired valve area comprises moving the distal tip toward an area of the mitral valve commissure on a side of the posterior papillary muscle, and further comprises subsequently operating the steerable introducer in a manner selected from the group of: (ii)(a) to move the distal tip toward the anterior leaflet of the mitral valve, rotating the flexible tubular catheter around its longitudinal axis in a counterclockwise direction when viewed distally, and (ii)(b) to move the distal tip toward the posterior leaflet of the mitral valve, rotating the flexible tubular catheter around its longitudinal axis in a clockwise direction when viewed distally.

20. A method as in claim 12, wherein the step of operating the steerable introducer to move the distal tip toward a desired valve area comprises moving the distal tip toward an area of the mitral valve commissure on a side of the posterior papillary muscle, and further comprises subsequently operating the steerable introducer in a manner selected from the group of: (ii)(a) to move the distal tip toward the anterior leaflet of the mitral valve, rotating the flexible tubular catheter around its longitudinal axis by about one-eighth turn in a counterclockwise direction when viewed distally, and (ii)(b) to move the distal tip toward the posterior leaflet of the mitral valve, rotating the flexible tubular catheter around its longitudinal axis by about one-eighth turn in a clockwise direction when viewed distally.
